(19) United States
(12) Patent Application Publication
(10) Pub. No.: US 2007/0078432 A1
(43) Pub. Date: Apr. 5, 2007

(54) Adjustable medication infusion injection apparatus

(76) Inventors: Thor R. Halseth, Agoura, CA (US); John M. Barker, Ventura, CA (US)

Correspondence Address: SANDRA LEE LIPKIN;A LAW CORPORATION, 1891 GOODYEAR AVE., SUITE 622, VENTURA, CA, 93003, US

(21) Appl. No.: 11/298,436

(22) Filed: Dec. 9, 2005

Related U.S. Application Data
(63) Continuation-in-part of application No. 11/221,246, filed on Sep. 6, 2005.

(51) Int. Cl. A61M 31/00

(52) U.S. Cl. 604/500

(57) ABSTRACT

An adjustable infusion injection apparatus which is designed to be used in conjunction with a surgically implanted infusate injection port. The apparatus utilizes a catheter assembly and an introducer needle assembly. The catheter assembly is to be installed on the skin of the patient in alignment with the port and the introducer needle is to be inserted in conjunction with the catheter assembly to insert a catheter within an internal chamber of the port and then the introducer needle is then removed with the catheter to then be used to inject medication from an inlet tube. During installation a movable portion of said catheter assembly can be moved to abuttingly be in contact with the skin of the patient.

### BACKGROUND OF THE INVENTION

1. Field of the Invention

This invention is directed generally to medical devices and more particularly to a device for accessing an infusion site of a person or animal and where the device is adjustable relative to the skin of the person or animal so that it can be placed in abutting contact with the skin.

2. Description of the Related Art

Introduction of liquid into a human or an animal is part of day-to-day medical practice within hospitals, clinics and doctors offices. Typical fluids comprise drugs, saline solution and chemotherapy. In the past, the injection of the drugs into the patient is obtained through a syringe with the needle of the syringe to be inserted into the patient and into a blood vessel, such as a vein. If the injection is quick requiring only a few seconds, the injection is accomplished by using a metallic needle. However in some instances, like for example, chemotherapy, the injection procedure may take a substantial period of time, such as an hour or more. Also within chemotherapy, the patient may require daily injection. This means that the injection has to be performed each day and may go on for weeks and even months.

The daily piercing of one's skin becomes an uncomfortable procedure for the patient. Also, most chemotherapy is quite toxic and inserting of such into a narrow vein located in an arm or a leg can actually damage the tissue wall of the vein. One of the ways in which chemotherapy can be injected to minimize toxic destruction of the vein tissue is by using of a surgically implanted infusate injection port. This port comprises a basic metallic cylindrical shaped housing that has an internal chamber which is closed on its upper surface by a rubberized septum. The internal chamber has a flexible conduit extending therefrom which is to be surgically implanted within a large diameter vein of the human or animal and within the human one of which is located in the area of the upper chest. This port is to be accessible repeatedly and a needle or a catheter can be inserted into the port through which medicaments can be supplied from the port into the blood vessel of the patient.

The use of such ports have many desirable features. The primary desirable feature is that the patient is not being stuck with a needle every day in order to administer chemotherapy. Additionally, another advantage is that the veins in some patients have a tendency to collapse easily and therefore by using a port this drawback of a collapsing vein is avoided. Also, utilizing a port decreases the amount of trauma to the patient's skin that would occur by being repeatedly stuck with a syringe.

In the past, a right angled Huber needle has been normally used to supply a medicament into a port. The needle is installed with the port and then taped to the patient and the patient may be free to move around for the period of time during administrating of the chemotherapy. The use of a metallic needle is not normally comfortable to a patient. It would be desirable, if possible, to utilize a flexible walled catheter instead of a metallic needle to supply medicament into the port. Flexible walled catheters are being used-instead of syringes for even the application of a medicament into a patient's arm or leg even when the infusion time is relatively short.

Reference is to be had to a prior filed patent application Ser. No. 11/221,246, filed Sep. 6, 2005, entitled, MEDICATION INFUSION INJECTION APPARATUS AND METHOD OF INSTALLING SAME, by the present inventors, which discloses a structure for installing of a flexible catheter within an infusate port. One of the disadvantages of this prior application is that at times the catheter apparatus does not abut against the skin of the patient when the catheter is fully installed within the port. It is spaced slightly from the skin. This spacing slightly from the skin makes it difficult to secure, as by taping, the catheter assembly onto the skin of the patient. It would be desirable to design the catheter to somehow be adjustable so that when the introducer needle, and its flexible catheter which is carried by the needle, has been installed in the correct position with the infusate port, the catheter assembly can be moved to be in abutting contact with the skin of the patient thereby facilitating the taping and fixing of the catheter assembly to the skin of the patient. Such an installation is not only more comfortable for the patient but protrudes to a lesser degree from the skin of the patient and is more stable when installed against the skin of the patient.

### SUMMARY OF THE INVENTION

The first basic embodiment of the present invention is directed to a method of installing an adjustable medication infusion injection apparatus in conjunction with a port surgically implanted through the skin of a patient comprising the steps of utilizing an introducer needle on which there is installed a catheter assembly where the catheter assembly has a flexible catheter which connects to a liquid inlet tube with liquid to flow through the inlet tube into the flexible catheter and with the tip of the needle protruding from the flexible catheter, inserting of the introducer needle through a septum of the surgically implanted port with the flexible catheter also being inserted into the port, moving of a portion of the catheter assembly relative to a remaining part of the catheter assembly and said introducer needle with said portion abutting the skin of a patient and withdrawing of the introducer needle leaving in position the catheter assembly.

A further embodiment of the present invention is where the first basic embodiment is modified by defining that the moving step is accomplished by means of a ratchet assembly included within the catheter assembly.

A further embodiment of the present invention is where the just previous embodiment is modified by defining that the moving step only permits movement of the portion in one direction toward the skin of the patient.

A second basic embodiment of the present invention is directed to an adjustable medication infusion injection apparatus adapted to be used with a surgically implanted infusate injection port comprising a catheter assembly and an introducer needle assembly. The introducer needle assembly includes an introducer needle which terminates at a sharpened tip. After inserting of a sharpened tip of the introducer needle through the port until the tip bottoms out, a portion of the catheter assembly is to be moved relative to the remaining part of the catheter assembly until the portion contacts the skin of the patient which surrounds the port. The introducer needle assembly is engagable with the catheter assembly for installing of a flexible catheter within the port. The introducer needle assembly is disengagable from the catheter assembly leaving the flexible catheter installed in the port.

A further embodiment of the present invention is where the second basic embodiment is modified by defining that the remaining part includes a linear toothed rail which is to be engagable by a pawl mounted on the portion of the catheter assembly.

A further embodiment of the present invention is where the just previous embodiment is modified by defining that the pawl is only movable on the rail in one direction.

A further embodiment of the present invention is where the second basic embodiment is modified by defining that the remaining part of the catheter assembly includes an open ended cylinder with the portion of the catheter assembly being slidably mounted within the cylinder.

A further embodiment of the present invention is where the second basic embodiment is modified by defining that there is an inlet conduit connected to the catheter assembly and medication is to be supplied through the inlet conduit into the catheter assembly and through the flexible catheter into the port.

A third basic embodiment of the present invention comprises an adjustable medication infusion injection apparatus which is adapted to be used with the surgically implanted infusate injection port. The injection apparatus comprises a catheter assembly and an introducer needle assembly. The catheter assembly includes a fixation platform mounted on a tubular housing. The tubular housing has an internal chamber. A piston is movably mounted in the internal chamber. A ratchet assembly interconnects the piston and the tubular housing. The ratchet assembly permits movement of the piston in one direction but prevents movement of the piston in an opposite direction. The fixation platform adapted to be taped onto the skin surrounding the port. A flexible catheter is fixedly attached to the fixation platform. The introducer needle assembly includes a needle having a sharpened tip, this tip to be inserted into the port which will carry with it the flexible catheter. The needle is to be withdrawn leaving the flexible catheter installed in the port.

A further embodiment of the present invention is where the third basic embodiment is modified by defining that the ratchet assembly comprises a linear toothed rail mounted on the tubular housing which is engaged with a pawl mounted on the piston.

### BRIEF DESCRIPTION OF THE DRAWINGS

For a better understanding of the present invention, reference is to be made to the accompanying drawings. It is to be understood that the present invention is not limited to the precise arrangement shown in the drawings.

FIG. 1 is an isometric view of the adjustable medication infusion injection apparatus of the present invention showing the apparatus in the position that it would be during installation in conjunction with a surgically implanted port;

FIG. 2 is an isometric view similar to FIG. 1 but depicting the apparatus in an adjusted position;

FIG. 3 is a view generally taken along line 3-3 of FIG. 1 showing the ratchet assembly that is utilized to affect movement of the apparatus of the present invention showing the ratchet assembly in a locked position;

FIG. 4 is a cross-sectional view similar to FIG. 3 but showing the ratchet assembly in a moving position;

FIG. 5 is a top plan view of the apparatus of this invention taken along line 5-5 of FIG. 2;

FIG. 6 is a longitudinal cross-sectional view through the apparatus of the present invention showing how such is installed in conjunction with a surgically implanted port where the introducer needle is located in conjunction with the port; and

FIG. 7 is a view similar to FIG. 6 but where the introducer needle has been removed.

### DETAILED DESCRIPTION OF THE INVENTION

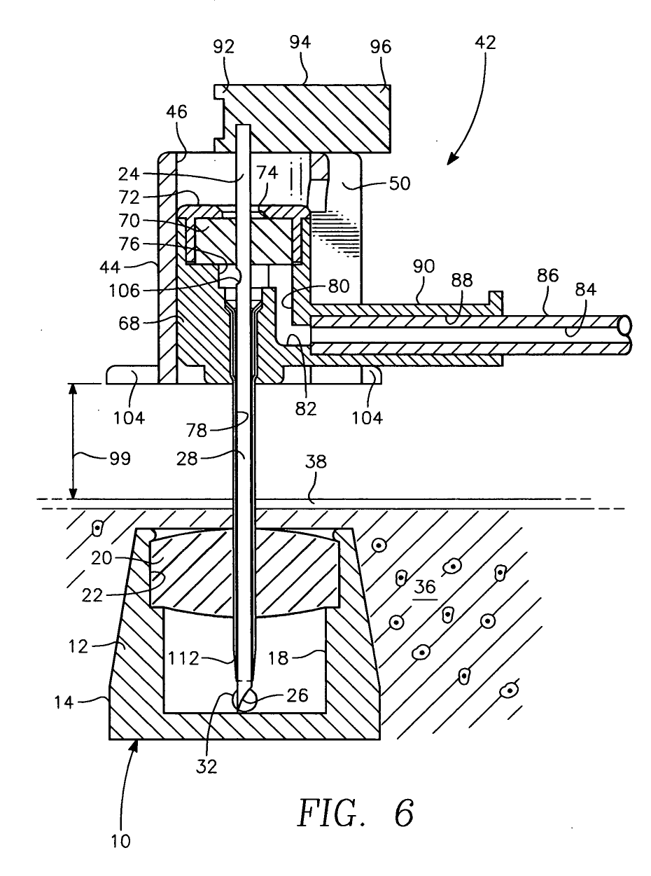

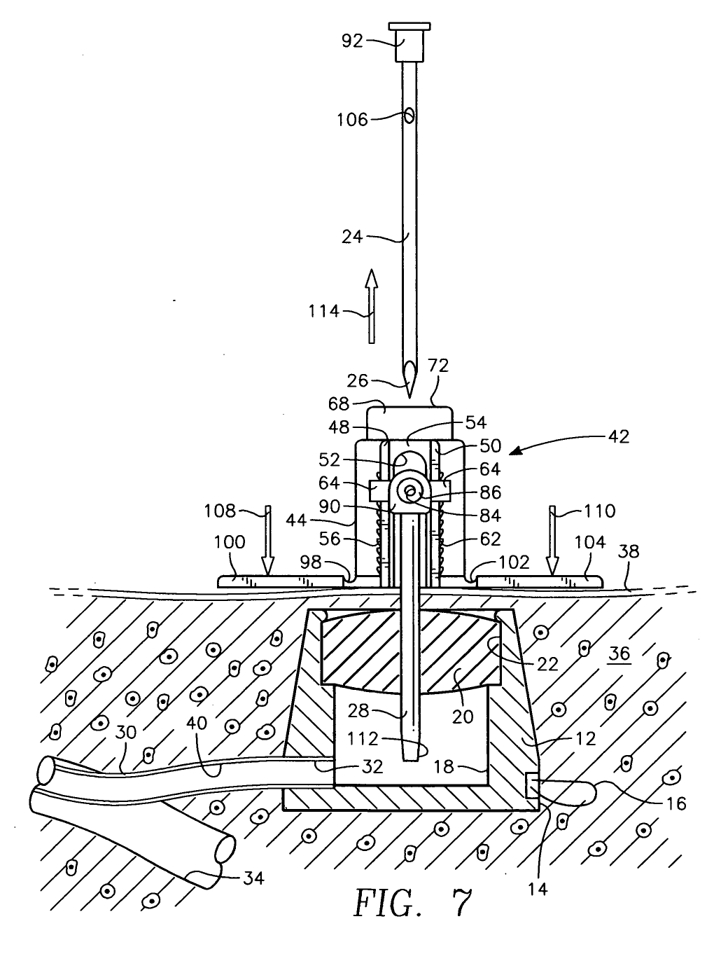

Referring particular to FIGS. 6 and 7 of the drawings, there is shown an infusate injection port 10 which is designed to be surgically implanted within the body of a human. It is considered to be within the scope of this invention that the port 10 could be implanted within non-humans, such as dogs and cats, but it is believed that the ports 10 are not commonly used in animals.

Housing 12 of port 10 is to be constructed of titanium or other inert metal. The housing 12 includes a base ring 14. Mounted within the base ring 14 are a plurality of threaded loops 16. It is to be noted that there are about eight in number of the threaded loops 16 evenly spaced apart, but the threaded loops 16 can be increased or decreased in number. The threaded loops 16 are to be used when surgically implanting of the port 10 within a patient to fix in position the port 10 within the patient's body.

The port 10 includes an internal chamber 18. This internal chamber 18 is closed by means of a rubberized septum 20 which is fixedly mounted in position with an annular recess 22 formed within the housing 12. The rubberized septum 20 is adapted to be penetrated by a sharp pointed instrument, such as an introducer needle 24. The introducer needle 24 has a sharpened tip 26. The introducer needle 24 is clearly shown in FIG. 7. The rubberized septum 20 closes the hole that was produced by the needle 24 and tightly binds against the wall surface of a flexible catheter 28 which is constructed of plastic. The introducer needle 24 is to be inserted within the flexible catheter 28 with the sharpened tip 26 protruding exteriorly of the flexible catheter 28.

The internal chamber 18 communicates with a flexible conduit 30.

Flexible conduit 30 is mounted within hole 32 formed within the housing 12. The flexible conduit 30 comprises generally a plastic tube with this conduit 30 to be surgically implanted within a blood vessel, such as a vein 34, shown in FIG. 7. The vein 34 is located in conjunction with the fatty tissue 36 of the human. The outer layer 38 of the skin of the human is placed over to cover the septum 20, as is clearly shown in FIG. 7 of the drawings. The lumen 40 of the conduit 30 is constantly open and connects directly to the internal chamber 18.

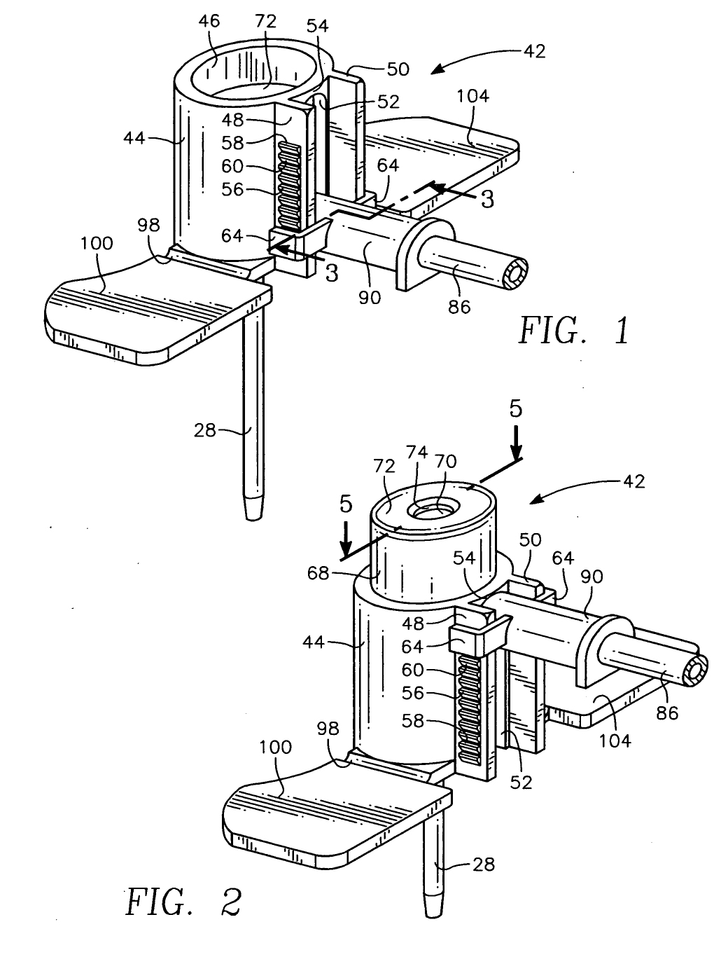

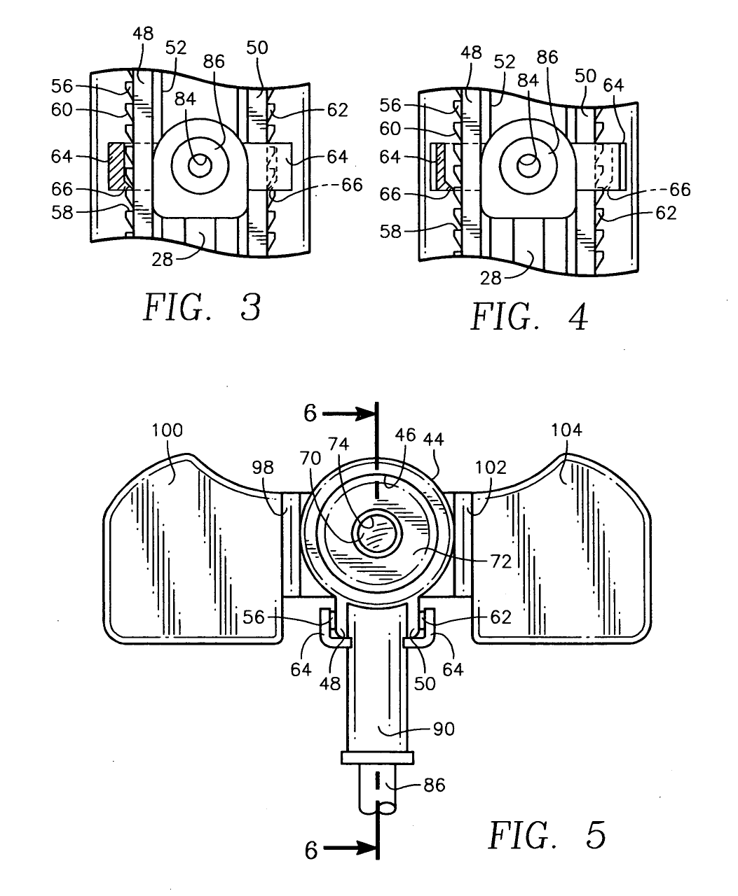

The medication infusion apparatus 42 includes a cylindrically shaped sleeve 44. The sleeve 44 has an internal chamber 46 which is open at each end. The sleeve 44 is integrally connected to a pair of longitudinal flanges 48 and 50. Formed within the sleeve 44 and located between the flanges 40 and 50 is an elongated slot 52. The slot 52 is closed by web 54 at its upper end but is open at its bottom end. The slot 52 provides access into the internal chamber 46. Mounted on the exterior surface of the flange 48 is a linear rail 56 which is formed of a plurality of evenly spaced apart ridge members each of which has an upper straight wall 58 and a lower slanted wall 60. A similar linear rail 62 is formed on the exterior surface of the flange 50. A yoke 64 is U-shaped and defines a pair of spaced apart legs with a connecting apex section. The inner surface of each of the legs of the yoke 64 include a pawl 66 (see FIG. 4). Each of the pawls 66 basically comprise inwardly extending protuberances formed on the inner surfaces of the legs of the yoke 64. Pawl 66 on one of the legs of the yoke 64 is to engage with the linear rail 56 and the opposite pawl 66 is to engage with the linear rail 62. The yoke 64 is snugly mounted relative to the flanges 48 and 50. It is to be noted that the sleeve 44 is capable of being moved from the position shown in FIG. 1 to the position shown in FIG. 2 or any position there between, and while it is being moved the pawls 68 ride up and over the ridges of the linear rail 56 by riding up the slanted walls 60 with the slanted walls 60 each functioning like a cam. However, once pawl 66 had ridden over a ridge, it is not capable of being moved in the reverse direction as the straight wall 58 of the ridge that has just been passed will keep that from happening.

A piston 68 is slidably mounted within the internal chamber 46. The piston 68 includes a rubberized septum 70 which is mounted in place on the piston 68 by a cap 72. The cap 72 has a center hole 74.

The inside surface of the septum 70 forms a closing surface for a fluid chamber 76 which is formed within the piston 68. This chamber 76 connects with a lumen 78 of the flexible catheter 28. The inner end of the flexible catheter 28 is fixedly mounted to the piston 68. The fluid chamber 76 connects by passageway 80 to a side entrance passage 82 which is formed through the sidewall of the piston 68. This passage 82 connects with lumen 84 of an inlet tube 86. The inlet tube 86 is tightly mounted within elongated hole 88 which is formed within a connecting sleeve 90. The connecting sleeve 90 is integral with the piston 68 and extends through the elongated slot 52.

The introducer needle 24 is fixedly mounted within a head 92. Fixedly mounted to the head 92 is a pin 94. The pin 94 is mounted within a handle 96. The handle 96 is to facilitate manual insertion and removal of the introducer needle 24.

Integrally connected by a “living hinge” 98 to its sleeve 44 is a left wing 100. Integrally connected by a “living hinge” 102 is a right wing 104. Wings 100 and 104 comprise a fixation platform. The wings 102 and 104 are capable of pivotal movement from the outwardly extending and usage position, shown in FIGS. 1 and 2, to a retracted position abutting against the sleeve 44, which is not shown.

The operation of the apparatus 42 of this invention is as follows. The medical practitioner is to take the apparatus 10 with the wings 100 and 104 in the retracted position up against the sleeve 44. The yoke 64 is at located at the lower end of the rails 56 and 62, which is shown in FIG. 1. The piston 68 is located adjacent the bottom end of the sleeve 44, which is shown in FIG. 6. The medical practitioner will take the introducer needle 24, insert such through the rubberized septum 70, and within the lumen 78 of the catheter 28 until the sharpened tip 26 extends exteriorly of the flexible catheter 28. This insertion of the introducer needle 24 is facilitated by means of the handle 96. The introducer needle 24 is conducted through the center hole 34, through the fluid chamber 76 in order to be located within lumen 78.

It is to be noted that the introducer needle 24 has a hole 106 formed within its sidewall. When the handle 96 abuts against the upper edge of the sleeve 44, as shown in FIG. 6, the apparatus 42 of this invention is ready for insertion in conjunction with the port 10. The medical practitioner folds the wings 100 and 104 in an outwardly extending position where the wings 100 and 104 are in alignment with each other, as is shown in FIGS. 1, 2, 5 and 7 of the drawings. The medical practitioner with one hand grabs onto the handle 96 and with the other hand applies pressure in the direction of arrows 108 and 110 on the wings 100 and 104 at the same time locating of the sharpened tip 26 of the introducer needle 24 exteriorly of the outer layer 38 of the skin directly adjacent the rubberized septum 20 of the port 10. Applying pressure pushing the sharpened tip 26 through the outer layer 38, through the rubberized septum 20 with this pressure to be continually applied until the medical practitioner “feels” that the sharpened tip has bottomed out against the internal chamber of the port 10. The medical practitioner then withdraws the introducer needle 24 by pulling outwardly on the handle 96, which is shown in FIG. 7. The rubberized septum 20 will snugly restrain the flexible catheter 28 and keep such installed in conjunction with the rubberized septum 20, as shown in FIG. 7, with the lower end 112 of the flexible catheter 28 located spaced from the bottom wall of the internal chamber 18. This is clearly shown in FIG. 7. The correct installation for the flexible catheter 28 has now been achieved.

In most instances, the result will be that the wings 100 and 104 are 5 some spaced distance, shown by arrow 99 from the outer layer 38, as is shown in FIG. 6. Wings 100 and 104, sleeve 44, connecting sleeve 70, yoke 6 and pawls 66 constitute a movable portion of the apparatus 42. The medical practitioner will grab onto connecting sleeve 90 and hold such in a fixed position while at the same time applying downward pressure in the direction of arrows 108 and 110 on the wings 100 and 104 respectively. This will cause a ratcheting to occur by the ridges of the linear rails 56 and 62 to be conducted over their respective pawls 68, as is clearly shown in FIG. 4 of the drawings. This conducting over is permitted by the legs of the yoke 44 slightly expanding outward. The wings 100 and 104 are to be continually moved downward until such come into abutting contact with the outer layer 38. This position for the movable portion is clearly shown in FIG. 7. The remaining part of the apparatus 42 constitutes the fixed part. The medical practitioner will then now tape the left wing 100 and the right wing 104 onto the outer layer 38 thereby fixing the apparatus 42 in position. The introducer needle 24, after it has been removed from the apparatus 42 as indicated by the directional arrow 114, is now to be discarded. Fluid is now to be conducted through the lumen 84, through passage 80 into fluid chamber 76. The fluid will now be conducted through hole 106, through the needle 24 to be discharged in the area of the sharpened tip 26 into the internal chamber 18 of the port 10. From the port 10 the fluid is to be conducted through the flexible conduit 30 into the vein 34.

Extraction of the apparatus 42 from the port 10 can be accomplished, when desired, by merely grabbing onto the sleeve 44 and pulling such in an outward direction which will disengage the flexible catheter 28 from the rubberized septum 20 and the hole that was formed within the rubberized septum 20 will then close.

The discussion included in this patent is intended to serve as a basic description. The reader should be aware that the specific discussion may not explicitly describe all embodiments possible and alternatives are implicit. Also, this discussion may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be added for the device described, but also a method claim is added to address the method of making the invention. It should also be understood that a variety of changes may be made without departing from the essence of the invention. Such changes are also implicitly included in the description. These changes still fall within the scope of this invention.

Further, each of the various elements of the invention and claims may also be achieved in a variety of manners. This disclosure should be understood to encompass each such variation, be it a variation of any apparatus embodiment, a method embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. It should be understood that all actions may be expressed as a means for taking that action or as an element which causes that action. Similarly, each physical element disclosed should be understood to encompass a disclosure of the action which that physical element facilitates. Such changes and alternative terms are to be understood to be explicitly included in the description.

What is claimed is:

1. A method of installing an adjustable medication infusion injection apparatus in conjunction with a port surgically implanted through the skin of a patient comprising the steps of:
utilizing an introducer needle on which there is installed a catheter assembly where said catheter assembly has a flexible catheter which connects to a liquid inlet tube with liquid to flow through said inlet tube into said flexible catheter and with a tip of said introducer needle protruding from said flexible catheter;
inserting of said introducer needle through a septum of said surgically implanted port with said flexible catheter also being inserted into said port;
moving of a movable portion of said catheter assembly relative to a remaining part of said catheter assembly and said introducer needle until said movable portion abuts the skin of a patient; and
withdrawing of said introducer needle leaving in position said catheter assembly.

2. The method as defined in claim 1 wherein said moving step is accomplished by means of a ratchet assembly included within said catheter assembly.

3. The method as defined in claim 2 wherein said moving step permits only movement of said movable portion in one direction toward the skin of a patient.

4. An adjustable medication infusion injection apparatus adapted to be used with a surgically implanted infusate injection port comprising:
a catheter assembly and an introducer needle assembly, said introducer needle assembly includes an introducer needle which terminates at a sharpened tip, after inserting of said introducer needle through said port until said tip bottoms out a movable portion of said catheter assembly is to be moved relative to the remaining part of said catheter assembly until said movable portion contacts skin of a patient which surrounds the port, said introducer needle assembly being engagable with said catheter assembly for installing of a flexible catheter within the port, said introducer needle assembly being disengagable from said catheter assembly leaving said flexible catheter installed in the port.

5. The adjustable medication infusion injection apparatus as defined in claim 4 wherein:
said remaining part includes at least one linear toothed rail which is to be engagable by a pawl mounted on said movable portion.

6. The adjustable medication infusion injection apparatus as defined in claim 5 wherein:
said pawl being movable on said rail in one direction.

7. The adjustable medication infusion injection apparatus as defined in claim 4 wherein:
said remaining part including an open ended cylinder, said movable portion being slidably mounted within said cylinder.

8. The adjustable medication infusion injection apparatus as defined in claim 4 wherein:
an inlet conduit connected to said catheter assembly, medication is to be supplied through said inlet conduit into said catheter assembly and through said flexible catheter into the port.

9. An adjustable medication infusion injection apparatus adapted to be used with a surgically implanted infusate injection port comprising:
a catheter assembly and an introducer needle assembly;
said catheter assembly including:
a fixation platform mounted on a sleeve, said sleeve having an internal chamber, a piston mounted in said internal chamber, a ratchet assembly interconnecting said piston and said sleeve, said ratchet assembly permits movement of said fixation platform relative to said piston in one direction but prevents movement of said fixation platform in the opposite direction, said fixation platform adapted to be taped onto the skin surrounding the port, a flexible catheter fixedly attached to said piston; and
an introducer needle assembly including:
a needle having a sharpened tip, said sharpened tip to be inserted into the port which will carry with it said flexible catheter, said needle then to be withdrawn leaving said flexible catheter installed in the port.

10. The adjustable medication infusion injection apparatus as defined in claim 9 wherein:
said ratchet assembly comprising a linear toothed rail mounted on said sleeve which is engaged with a pawl which is fixed relative to said piston.
